(19) United States
(12) Patent Application Publication
(10) Pub. No.: US 2021/0130789 A1
(43) Pub. Date: May 6, 2021

(54) METHODS OF STROMAL CELL EXPANSION, USES AND MATERIALS RELATED THERETO

(71) Applicant: CITY OF HOPE, Duarte, CA (US)

(72) Inventors: Ismail H. AL-ABDULLAH, Azusa, CA (US); Meirigeng QI, Duarte, CA (US); Hsun Terasa KU, Duarte, CA (US); Bashar KHIATAH, Duarte, CA (US)

(73) Assignee: CITY OF HOPE, Duarte, CA (US)

(21) Appl. No.: 17/082,642

(22) Filed: Oct. 28, 2020

Related U.S. Application Data
(60) Provisional application No. 62/928,308, filed on Oct. 30, 2019.

(51) Int. Cl. C12N 5/0775; C12N 5/071

(52) U.S. Cl. CPC C12N 5/0668; C12N 5/0613; C12N 2501/148; C12N 2500/32; C12N 2500/38

(57) ABSTRACT

Disclosed herein are methods for expanding and potentiating intra-pancreatic tissue-derived mesenchymal stromal/stem cells in the presence of TNF-α, DMOG, or both, to obtain IPTD-MSCs having enhanced pro-angiogenic and/or anti-inflammatory properties. Also disclosed are therapeutic applications of these IPTD-MSCs.

### PRIORITY CLAIM

This application claims priority to U.S. Patent Application No. 62/928,308, filed Oct. 30, 2019, which is incorporated herein by reference in its entirety, including drawings.

### BACKGROUND

Mesenchymal stromal cell (MSCs) have the potential for treating various diseases [1]. Currently, over eight hundred clinical trials involving MSCs have been registered (clinicaltrials.gov), and the majority of which are focusing on application of MSCs to diseases of the musculoskeletal and cardiovascular systems as well as autoimmune type 1 diabetes (T1D) [2, 3]. With respect to the treatment of diabetes with MSCs, some encouraging progresses have been made. For example, intravenous injection of umbilical blood-derived allogeneic MSCs improved the function of pancreatic β-cells, reduced the incidence of diabetic complications and led to insulin independence in some type 2 diabetic patients [4, 5]. Autologous MSCs were used to treat individuals with T1D and lead to preservation of C-peptide [6]. For this, bone marrow-derived MSCs were aspirated from iliac crest aspiration, a procedure with substantial discomfort [6]. Moreover, administration of bone marrow-derived allogeneic MSCs together with pancreatic islets enhanced islet survival in diabetic non-human primates [7]. These studies employed fetal bovine serum in the MSC culture media, which is less desirable than media that lack animal proteins, pointing to a need for alternative culture and expansion strategies. The technology disclosed herein meets a need in the art by providing an improved source of MSCs and/or islets as well as improved methods and materials for producing MSCs and/or islets for any use.

### SUMMARY

In certain embodiments, conditioning protocols for a primary cell culture are provided, where such protocols include steps of culturing a population of primary cells isolated from a donor tissue or organ in a primary cell culture medium and treating the population of primary cells with dimethyloxalylglycine (DMOG), tumor necrosis factor α (TNF-α), or both DMOG and TNFα by adding the DMOG and/or TNFα to the primary cell culture medium. In some embodiments, the primary cell culture medium comprises a base cell culture medium supplemented with human platelet lysate (hPL), vitamin C, glutathione or a combination thereof.

In certain embodiments, methods of generating one or more populations of primary cells for therapeutic application, where such methods include steps of isolating a first population of primary cells from a donor tissue or organ, and performing a conditioning protocol (e.g., the conditioning protocol described above) on the first population of primary cells. Such methods may also include steps of isolating a second population of primary cells from a donor tissue or organ, and performing the conditioning protocol on the second population of primary cells. In certain aspects, the populations of primary cells that may be used in the embodiments described herein may be a population of mesenchymal stem cells and/or a population of islets.

In certain embodiments, methods of generating a mixture of cells for islet transplantation, where such methods include isolating a population IPTD-MSCs and a population of islet cells from a single donor pancreas, separately performing a conditioning protocol (e.g., the conditioning protocol described above) on the population of IPTD-MSCs and the population of islet cells, and combining the conditioned IPTD-MSCs and the islet cells to form the mixture of cells for transplanting into a subject.

In certain embodiments, methods for treating a disease or condition (e.g., type 1 diabetes) comprising co-transplanting a therapeutically effective amount of a mixture of a population IPTD-MSCs and a population of islet cells from a single donor pancreas or pancreata.

### BRIEF DESCRIPTION OF THE DRAWINGS

This application contains at least one drawing executed in color. Copies of this application with color drawing(s) will be provided by the Office upon request and payment of the necessary fees.

FIG. 1 illustrates a method for isolating intra-pancreatic tissue-derived (IPTD) cells from digested human pancreatic tissue. A schematic diagram showing the steps for isolating in a cGMP facility intra-pancreatic tissue-derived cells during human islet isolation.

FIGS. 2A-2C show that IPTD cells resembled MSCs in culture and could be cryopreserved. Phase contrast microscopy of IPTD-MSCs cells cultured in CMRL-1066 medium supplemented with 5% hPL. FIG. 2A: Passage 3 cell culture on day 3. FIG. 2B: Passage 5 cell culture on day 3. FIG. 2C: Passage 3 cells after 9 months of cryopreservation, thawing and culture on day 3. Representative images are presented. Scale bar, 200 μm.

FIGS. 3A-3B show that the growth of IPTD-MSCs was enhanced by a culture medium supplemented with hPL. FIG. 3A: Total cell numbers during expansion under the designated culture conditions. hPL was essential for the expansion of cells in vitro. The results shown were from three different tissue donors. FIG. 3B: A representative photomicrograph of passage 3 cells cultured in CMRL-1066 culture medium with (green) and without (red) 5% hPL. Scale bar, 100 μm.

FIGS. 4A-4B show that IPTD-MSCs display a cell-surface protein profile consistent with classic MSCs. FIG. 4A: Flow cytometry analysis of cell-surface protein expression of bone marrow-derived MSCs and IPTD-MSCs. Data are representative of four pancreas donors. FIG. 4B: Immunofluorescent staining for CD105 protein expression (green) in paraffin sections of IPTD-MSCs (passage 3 and 5) grown in the presence of hPL. Scale bar=50 μm. DAPI stains nuclei.

FIG. 5 shows that cells expressing CD105 were found in non-digested pancreatic tissue. Double immunofluorescent staining for CD105 (green) and insulin (red) revealed that CD105 positive cells were present in the pancreatic tissue and located adjacent to the insulin-expressing islets. Photomicrographs were obtained using a Z1 microscope (Carl Zeiss) at florescence wavelengths of 488 nm (CD 105) and 647 nm (insulin). Images are representative of 3 separate experiments. Scale bars, 50 μm.

FIGS. 6A-6E show that IPTD-MSCs could be differentiated into adipogenic, chondrogenic, and osteogenic cells. FIG. 6A: Passage 3 of IPTD-MSCs prior to differentiation. FIG. 6B: Lipid droplets were detected after staining with Oil Red O indicating that IPTD-MSCs were undergoing adipogenic differentiation 21 days post culture. FIG. 6C: The presence of cartilage was confirmed after staining with Alcian Blue showing in dark blue color. FIG. 6D: Calcium deposition was detected in paraffin-fixed tissue section of the cells cultured in chondrogenic media after staining with Alcian Blue. FIG. 6E: Calcium deposition was detected in paraffin-fixed tissue section of the cells cultured in osteogenic medium after staining with von Kossa.

FIGS. 7A-7C show that IPTD-MSCs treated with TNF-α and DMOG displayed increased mRNA levels of anti-inflammatory and pro-angiogenic genes. FIG. 7A (TSG-6), FIG. 7B (NRF2) and FIG. 7C (VEGF) mRNA levels from IPTD-MSCs treated with TNF-α (10 ng/mL) and/or DMOG (1 mM). Three independent donors were tested. The data are expressed as mean±SEM. *p<0.05; **p<0.01; ***p<0.001; ****p<0.0001.

FIG. 8 shows that IPTD-MSCs treated with TNF-α and DMOG produced increased levels of several cytokines. Relative changes of the indicated cytokines and growth factors found in medium from conditioned and control IPTD-MSCs. Y-axis is logarithmic. Four independent experiment were performed. The data are expressed as mean±SEM. *p<0.05; **p<0.01; ***p<0.001; ****p<0.0001; n.s. not significant.

FIGS. 9A-9B show that IPTD-MSC-derived media stimulated endothelial tube formation. FIG. 9A: Representative photographs of endothelial cells cultured and treated with medium from conditioned and control IPTD-MSCs at 4 hours and 24 hours post-culture. Scale bar, 500 μm. FIG. 9B: Quantification is presented as total tube number and total tube length after 24 hours. Duplicate samples were performed. Representative microscopic images are presented.

FIG. 10 shows that human islets (Fr 1, purity >70%) treated with DMOG (1 mM) displayed increased mRNA levels of VEGF gene. Four independent donors were tested. The data are expressed as mean±SD. **p<0.01.

FIG. 11 shows that human islets (Fr 2, purity <50%) treated with DMOG (1 mM) displayed increased mRNA levels of INS, PDX1, TSG-6, VEGF, and EPO genes. Six independent donors were tested. The data are expressed as mean±SD. *p<0.05; **p<0.01; ****p<0.0001.

FIG. 12 shows that pancreatic ductal cells treated with DMOG (1 mM) displayed increased mRNA levels of VEGF and TSG-6 genes. One donor was tested. The data are expressed as mean±SD.

### DETAILED DESCRIPTION

Disclosed herein are methods of isolating and culturing primary cells, including mesenchymal stem cells from any source and intra-pancreatic tissue-derived (IPTD)-MSCs from tissue fractions that are routinely discarded during pancreatic islet isolation of human cadaveric donors. Also disclosed are culture methods and materials for enhancing pro-angiogenic and anti-inflammatory properties of these and other cells.

Methods for Culturing Primary Cells and Generating Primary Cells for Therapeutic Application

According to the embodiments described herein, methods for culturing primary cells isolated from a donor tissue or organ are provided. The primary cells may be any type of cell that can be isolated from a suitable donor organ or tissue, and then can be expanded, modified, and/or conditioned to generate therapeutic cells for transplanting into a recipient for the treatment or prevention of a disease or condition.

The isolated primary cells may be expanded through one or more passages, and subsequently subjected to a conditioning protocol according to certain embodiments to simplify and/or improve the success of subsequent transplantation. After expansion, the primary cells may be divided into one or more aliquots and cryopreserved before being subjected to the conditioning protocol according to some embodiments.

In certain embodiments, the conditioning protocol may include a method that includes a step of culturing a population or aliquot of primary cells isolated from a donor tissue or organ in a primary cell culture medium. The primary cell culture medium is a GMP-compatible medium that does not contain animal protein or other xenobiotic components. In certain embodiments, the primary cell culture medium includes a base cell culture medium supplemented with one or more additional components.

The base cell culture medium may be prepared using any suitable ingredients for primary cell growth or may be obtained from any suitable commercial source. Non-limiting examples of base cell culture medium include, but are not limited to, Eagle's Minimum Essential Medium (EMEM), Dulbecco's Modified Eagle's Medium (DMEM), RPMI-1640, Ham's nutrient mixtures (F-10, F-12), Iscove's Modified Dulbecco's Medium (IMDM), CMRL, Glasgow Minimum Essential Medium (G-MEM), Leibovitz's L-15, CcCoy's 5A, BGJb, BME, Brinster's BMOC-3, MCDB, Waymouth's MB 752/1, or Williams' Media E.

The base cell culture medium may be supplemented with one or more of the following components: human platelet lysate (hPL), vitamin C, glutathione, or other growth factors such as bFGF. In certain embodiments, the primary cell culture medium includes a base cell culture medium supplemented with a concentration of hPL. In other embodiments, the primary cell culture medium includes a base cell culture medium supplemented with a concentration of vitamin C. In other embodiments, the primary cell culture medium includes a base cell culture medium supplemented with a concentration of glutathione. In other embodiments, the primary cell culture medium includes a base cell culture medium supplemented with a concentration of hPL and vitamin C. In other embodiments, the primary cell culture medium includes a base cell culture medium supplemented with a concentration of hPL and glutathione. In other embodiments, the primary cell culture medium includes a base cell culture medium supplemented with a concentration of vitamin C and glutathione. In other embodiments, the primary cell culture medium includes a base cell culture medium supplemented with a concentration of hPL, vitamin C, and glutathione.

In embodiments where the primary cell culture medium includes hPL, the hPL is added to the base cell culture medium at a concentration of approximately 2% or higher, 3% or higher, 4% or higher, 5% or higher, 6% of higher, 7% or higher, 8% or higher, or between 2.5% to 8% according to some embodiments. In one embodiment, hPL is added to the base cell culture medium at a concentration of approximately 5%.

In embodiments where the primary cell culture medium includes vitamin C, the vitamin C is added to the base cell culture medium at a concentration of approximately 2 mM to 4 mM according to some embodiments. In one embodiment, vitamin C is added to the base cell culture medium at a concentration of approximately 3 mM. In some embodiments, the vitamin C added to the base cell culture medium is a stable form of vitamin C, e.g., 2-O-alpha-D-glucopyranosyl-L-Ascorbic acid.

In certain embodiments where the primary cell culture medium includes glutathione, the glutathione is added to the base cell culture medium at a concentration of approximately 8 mg/L to 12 mg/L according to some embodiments. In one embodiment, glutathione is added to the base cell culture medium at a concentration of approximately 10 mg/L.

In certain embodiments a kit that includes a first container including the primary cell culture medium, a second container including a solution of DMOG, and a third container including a solution of TNF-α is provided. The contents of the container may be supplied in solution, or may be supplied in powder or other stable forms for reconstitution upon purchase of the kit.

The conditioning protocol may also include a step of treating the primary cells with dimethyloxallyl glycine (DMOG), tumor necrosis factor α (TNF-α), or both DMOG and TNFα by adding the DMOG and/or TNFα to the base cell culture medium. In some embodiments, the DMOG is added at a concentration of 1 mM, but may be added at any suitable nontoxic concentration to produce the same effects. In other embodiments, the TNF-α is added at a concentration of 10 ng/ml, but may be added at any suitable nontoxic concentration to produce the same effects.

In some embodiments, the primary cells may be cultured to a desired confluence prior to treatment with DMOG and/or TNFα. For example, the primary cells may be cultured to approximately 50% or higher confluence, approximately, 50%, 60%, 70%, 80%, 90%, higher than 90% confluence, or fully confluent. In some embodiments, the time of treatment may be any suitable time period to achieve desired characteristics (e.g., increased expression of TSG-6, and/or NRF2, and/or VEGF), and in certain embodiments, the primary cells are treated with DMOG and/or TNFα for approximately 24 to 48 hours.

The conditioning protocols described herein may be used in methods for generating one or more populations of primary cells for therapeutic applications. In such embodiments, the method may include steps of isolating a first population of primary cells from a donor tissue or organ and, optionally, isolating a second population of primary cells from a donor tissue or organ. In some embodiments, the first and second populations of primary cells are isolated from the same donor tissue or organ. In other embodiments, the first and second populations of primary cells are isolated from different donor tissues or organ (i.e., isolated from a first and second donor tissue or organ, respectively). Isolating the first and second populations of primary cells from the donor tissue(s) or organ(s) may be performed using suitable techniques known in the art for establishing primary cell culture for transplantation, and each of the first and second populations of primary cells are cultured separately. In some embodiments, each of the first and second populations of primary cells may be expanded in a primary cell culture described herein. Following isolation and optional expansion, the conditioning protocol described above may be performed on the first and second populations of primary cells to generate the one or more populations of primary cells for therapeutic applications. Optionally, the first and second populations of primary cells may be cryopreserved in aliquots for storage prior to performing the conditioning protocol.

Suitable donor organs or tissues that may be used in the embodiments described herein may include, but are not limited to, blood, lymph nodes, bone marrow, endothelial tissue, epithelial tissue, muscular tissue, nervous tissue, connective tissue, adipose tissue, umbilical cord, umbilical cord blood, placenta, amniotic fluid, embryo, pancreas, liver, kidney, bones, heart, vascular tissue, stomach, intestines, lung, ocular limbus and skin. The donor organ or tissue may be derived from any suitable source. For example, the donor organ or tissue may be a cadaver donor organ or tissue, an autologous donor organ or tissue, or an allogenic donor organ or tissue.

Primary cells that can be isolated from the donor organ or tissue and used as the first and/or second populations of primary cells may include, but are not limited to, immune cells (e.g., B lymphocytes, T lymphocytes, monocytes, macrophages, natural killer cells, eosinophils, basophils, neutrophils), stem cells (e.g., embryonic stem cells, tissue-specific stem cells, mesenchymal stem cells, induced pluripotent stem cells), endothelial cells, epithelial cells, smooth muscle cells, skeletal muscle cells, cardiac muscle cells, neurons, glial cells (e.g., astrocytes, microglial cells, oligodendrocytes, ependymal cells), fibroblasts, mast cells, plasma cells, macrophages, adipocytes, leukocytes, erythrocytes, thrombocytes, chondrocytes, osteoblasts, osteoclasts, osteocytes, osteoprogenitor cells, islet cells, acinar cells, renal cells, cardiac conductive cells, epidermal cells (e.g., Merkel cells, keratinocytes, with melanocytes and Langerhans cells), and dermal cells.

In certain embodiments, the population of cells isolated from the donor organ or tissue is a population of mesenchymal stem cells (MSC). The MSCs isolated and conditioned as described herein may be used in an organ or tissue transplantation procedure and discussed in the embodiments herein. In other embodiments, the MSCs isolated and conditioned as described herein may be used alone or in combination with other cells or treatments to treat, prevent or cure various disease conditions such as rheumatoid arthritis (RA), systemic lupus erythematosus (SLE), or any other autoimmune diseases due to enhanced anti-inflammatory effects to surrounding cells Alternatively, the MSCs isolated and conditioned as described herein may be used to generate a cell line for use in in vitro research. For example, such a cell line may be used to in cell cultures for extracellular matrix development when co-cultured with other stem cells (e.g., embryonic stem cells, induced pluripotent stem cells) to facilitate the other stem cell growth and expansion.

In some embodiments, the primary cells are mesenchymal stem cells (MSC) isolated from donor bone marrow, blood, adipose tissue, lung, heart, umbilical cord, or placenta. In other embodiments, the primary cells are MSCs isolated from a donor pancreas. In other embodiments, the first and/or second populations of primary cells are MSCs and islet cells isolated from a donor pancreas. In other embodiments, the first population of primary cells are MSCs isolated from donor bone marrow, blood, adipose tissue, lung, heart, umbilical cord, or placenta and the second population of primary cells are islet cells isolated from a donor pancreas, and vice versa. The donor pancreas, bone marrow, blood, adipose tissue, lung, heart, umbilical cord, or placenta may be from a cadaver, an autologous donor, an allogenic donor, or a combination thereof. In one embodiment, MSCs and islet cells are isolated from a single donor pancreas. As described below, MSCs may be isolated from otherwise normally discarded fractions of pancreatic tissue. Harvesting multiple cell types from tissue fractions of a single donor organ is not only beneficial to simplify treatment procedures, but with respect to the previously under-appreciated fraction of pancreatic tissue that includes MSC, it also may uncover additional cell types that reside within that tissue fraction that may be used to study pancreatic pathophysiology of diseases in the pancreas or other organs.

The donor of the organ or tissue (and thus the primary cells) and the recipient of a transplant in accordance with the embodiments described herein may be a human or any other mammal (e.g., mice, rats, rabbits, dogs, cats, guinea pigs, hamsters, monkeys, apes, horses, pigs, cattle). The donor organ and isolated cells therefrom may be used in an interspecies transplant in certain embodiments, while in other embodiments, the donor organ and isolated cells therefrom may be used in an intraspecies transplant. As such, the methods may be applied in vivo, in vitro, or ex vivo in a medical or therapeutic setting, a veterinary setting, or in a research setting.

Methods for Generating Primary Cells from Pancreata for Islet Transplantation

The culture methods described herein may be used to generate one or more populations of primary cells that can be transplanted into a recipient to treat or prevent a disease or condition. In some embodiments, the culture methods described herein are used to generate a mixture of cells for islet transportation, which is discussed further below.

Islet transplantation has been shown to be an effective and safe cellular therapy for patients with brittle type 1 diabetes mellitus (T1DM) who have unstable glucose levels resulted into frequent episodes of hypoglycemia unawareness. This mode of therapy has been approved by national health system of Canada, Europe, Scandinavian countries and Australia as a standard procedure for treating T1DM. In the US, Food and Drug Administration (FDA) is evaluating such therapy for potential licensed treatment towards T1DM. However, sustained and long-term outcome of clinical islet transplantation remains challenging. This includes deterioration of islet mass and function, allogeneic rejection and/or autoimmune recurrence, and poor islet engraftment.

To overcome the limitations in islet transplantation, studies on mesenchymal stem cells (MSCs) have recently been escalated. For example, MSCs derived from tissues like bone marrow, adipose tissue, and umbilical cord have been investigated in the clinical setting for their immuno-modulatory and tissue regenerative properties. In the context of islet transplantation, MSCs derived from bone marrow have been tested to improve engraftment of pancreatic islets by suppressing inflammatory damage and immune-mediated rejection. Yet on the platform of clinical islet transplantation, co-transplantation of islets and MSCs originating from intrapancreatic tissue has never been successfully conducted. Therefore, disclosed herein is a method of isolating MSCs cells from donor pancreatic tissue (e.g., cadaveric pancreata) and expanding the isolated cells for therapeutic applications. These MSCs can be directly produced from a single organ source using methods described above during islet manufacturing process in GMP facilities, greatly facilitating and streamlining co-transplantation of islets and MSCs.

According to the embodiments described herein, methods for generating one or more populations of MSCs and islets for use in islet transplantation are provided. In such embodiments, the method may include steps of isolating a population of MSCs and a population of islets from donor tissue(s) or organ(s). In some embodiments, the MSCs and islets are isolated from a single donor pancreas or group of pancreata. In other embodiments, the MSCs and islets may be isolated from different donor tissues or organs. For example, in some embodiments, the population of MSCs is isolated from bone marrow, blood, adipose tissue, lung, heart, umbilical cord, or placenta, while the population of islets is isolated from a donor pancreas or pancreata. In the embodiments where the MSCs are isolated from a donor pancreas or group of pancreata, the MSCs may be referred to as intra-pancreatic tissue-derived (IPTD)-MSCs.

Isolating the first and second populations of MSCs (or IPTD-MSCs) and islets from the donor tissue(s) or organ(s) may be performed using suitable techniques known in the art for establishing primary cell culture for islet transplantation, then the MSCs (or IPTD-MSCs) and islets are cultured separately. In some embodiments, the MSCs (or IPTD-MSCs) and islets may be expanded in a primary cell culture described herein.

In one embodiment—described in detail in the working examples below—an isolation technique that may be used with the methods described herein is provided. During human islet isolation, stromal cells from cadaveric pancreata are simultaneously isolated after enzyme digestion. The stromal cells were characterized and found to be pure mesenchymal stem cells (MSCs) with the following surface markers: CD105, CD73, and CD90, but negative for CD45, CD34, and CD14. Investigators have isolated cells that present and absent with these cell surface markers from various tissues including bone marrow, umbilical cord blood, and adipose tissue. However, there is limited evidence in the literatures that these cells were also present within pancreatic tissue. As disclosed herein, MSCs are in fact present and can be expanded after cryopreserving the cells for more than a year with full recovery.

Following isolation and optional expansion, the conditioning protocol described above may be performed on the MSCs (or IPTD-MSCs) and islets to generate the cells for islet transplantation. Optionally, the MSCs (or IPTD-MSCs) and/or islets may be cryopreserved in aliquots for storage prior to performing the conditioning protocol.

Under normoxic condition (5% CO₂, 21% O₂), isolated IPTD-MSCs and islet cells do not express tumor necrosis factor-α-stimulated gene 6 (TSG6, a factor that has anti-inflammatory properties of cells) and vascular endothelial growth factor (VEGF). However, when these cells were cultured in the presence of dimethyloxalylglycine (DMOG) under normoxic condition, both TSG6 and VEGF were upregulated to 31- and 51-fold respectively. Surprisingly, when the MSC were cultured in the presence of TNF-α plus DMOG, TSG6 was upregulated to 70-fold, which was a drastic increase when compared to the TSG6 levels stimulated by TNF-α alone. This result indicated that the combination of DMOG and TNF-α could enhance anti-inflammatory effect to the cells. Furthermore, DMOG protected the human islets and MSC from hypoxia by inhibiting Hypoxia-inducible Factor Prolyl Hydroxylase (HIF-Phase) resulted into augmentation of certain transcription factor(s) associated that could be highly beneficial for the islet and cell survival and function. Islets in particular are oxygen sensitive and when oxygen demand exceeds the supply, the HIF pathway is triggered to adapt for hypoxic conditions as a result of inactivation of HIF-Phase, an enzyme that contributes to degradation of HIF-1α. Thus, inhibiting HIF-Phase enzyme activity leads to increased HIF-1α protein in cells. Up-regulation of HIF-1α at transcriptional and translational levels leads to production of erythropoietin (EPO) and other proteins, including glycolytic enzyme, glucose transporter-1 (Glut-1) and vascular endothelial growth factor (VEGF). These factors are important to protect islets from cytokine induced cell death and also potentiate islet function, survival, and proliferation.

Islet transplantation may be accomplished by any suitable administration or surgical method known in the art. In certain embodiments, islet (co-)transplantation may be accomplished by infusing or injecting islets and MSCs (or IPTD-MSCs) into the liver via a catheter into the portal vein.

In some embodiments, the MSCs (or IPTD-MSCs) and islets generated as discussed above may be combined or mixed together prior to islet transplantation. Thus, after performing the conditioning protocol on the MSCs (or IPTD-MSCs) and islets, the conditioned MSCs (or IPTD-MSCs) and the islet cells are combined to form the mixture of cells for co-transplanting the two populations of primary cells into a subject. Alternatively, the population of MSCs (or IPTD-MSCs) and the population of islets may be co-transplanted via separate subsequent infusions (one after the other) or via simultaneous infusions.

In certain embodiments, the cells and mixtures of cells generated as described above may be used in methods for treating a condition or disease related to impaired function and/or condition of the pancreas. Examples of conditions or diseases that may be treated using the methods described herein include, but are not limited to, type 1 diabetes, acute pancreatitis, chronic pancreatitis, benign or malignant pancreatic tumors, or any other condition in which islet transplantation is indicated. Such methods include a step of co-transplanting a therapeutically effective amount of MSCs (or IPTD-MSCs) and islets separately or as part of a mixture of cells described herein.

“Treating” or “treatment” of a disease or condition may refer to preventing the disease or condition, slowing the onset or rate of development of the disease or condition, reducing the risk of developing the disease or condition, preventing or delaying the development of symptoms associated with the disease or condition, reducing or ending symptoms associated with the disease or condition, generating a complete or partial regression or reversal of the condition, or some combination thereof.

The methods for treating the diseases and conditions described herein include transplantation or co-transplantation of a therapeutically effective amount of certain populations of cells and mixtures of such cells. An “effective amount,” “therapeutically effective amount” or “effective dose” is an amount of cells or group(s) of cells (e.g., individual cells or islets) that produce a desired therapeutic effect in a subject, such as preventing or treating a target condition or alleviating symptoms associated with the condition. The precise therapeutically effective amount is an amount of the composition that will yield the most effective results in terms of efficacy of treatment in a given subject. This amount will vary depending upon a variety of factors, including but not limited to the characteristics of the cells, the physiological condition of the subject (including age, sex, disease type and stage, general physical condition, responsiveness to a given dosage, and type of medication), the nature of any pharmaceutically acceptable carrier or carriers delivered with the cells, and the route of administration. One skilled in the art will be able to determine a therapeutically effective amount through routine experimentation, namely by monitoring a subject's response to administration of a compound and adjusting the dosage accordingly. For islet transplantation, the amount of islets transplanted to a subject is often expressed as islet equivalents, or IE (one IE is considered equivalent to a pancreatic islet with a diameter of 150 μm) per kilogram of recipient body weight. In some embodiments, a therapeutically effective amount of islets per transplantation procedure is approximately 4,000 IE/kg or more, and the total number of islets transfused over one or more transplantation procedure is between approximately 4,000 IE/kg and approximately 15,000 IE/kg. MSCs may be transplanted in an amount that acts as an adjuvant to islet function and that protects islet. In such embodiments, a therapeutically effective amount of islets may be lower than embodiments that do not include MSCs (i.e., lower than 4,000 IE/kg per transplantation procedure and/or total number of islets transplanted). In certain embodiments, MSCs may be co-transplanted with islets in an amount along the magnitude of approximately 10⁶-10⁷ cells.

As described in working examples below, several other embodiments are disclosed. In one embodiment, a method of expanding and potentiating ex vivo intra-pancreatic tissue-derived (IPTD) mesenchymal stem cells (MSCs) is provided. The method comprises the steps of isolating MSCs from pancreatic tissues, and culturing the isolated MSCs in a culturing medium containing TNF-α, dimethyloxallyl glycine (DMOG), or both, thereby to obtain the IPTD-MSCs, wherein the IPTD-MSCs have enhanced pro-angiogenic properties or enhanced anti-inflammatory properties compared with IPTD-MSCs cultured without TNF-α or DMOG. In some embodiments, the MSCs are isolated from pancreata. In some embodiments, the MSCs are isolated from human pancreata. In some embodiments, the culturing medium contains human platelet lysate, a stable form of vitamin C, glutathione, or a combination thereof. In some embodiments, the IPTD-MSCs obtained by the disclosed method are positive for CD90, CD105, and CD73, and negative for CD45, CD34, CD14, and HLA-DR. In some embodiments, at least 95% of the IPTD-MSCs obtained by the disclosed method are positive for CD90, CD105, and CD73, and negative for CD45, CD34, CD14, and HLA-DR. In some embodiments, the IPTD-MSCs obtained by the disclosed method are capable of differentiation into adipocytes, chondrocytes, and osteoblasts in vitro.

In another embodiment, a method of isolating IPTD-MSCs simultaneously along with harvesting islets from a single donor is provided. The method includes the steps of digesting isolated pancreas obtained from a single donor with an enzyme, separating the pancreas tissue such as islets and acinar cells from dissociated cells by centrifugation, purifying islets from the pancreas tissue and filtering the dissociated cells to obtain MSCs. The obtained MSCs can be further expanded and potentiated ex vivo using the methods disclosed above.

In another embodiment, a method of co-transplanting islets with IPTD-MSCs in a subject is provided. In some embodiments, the IPTD-MSCs are expanded and potentiated ex vivo using the methods disclosed above before co-transplanting with the islets to the subject. In some embodiments, the islets are potentiated ex vivo by treating with DMOG. In some embodiments, the islets and the IPTD-MSCs for co-transplantation are harvested simultaneously from a single donor.

In another aspect, disclosed is a method of treating type 1 diabetes mellitus (T1DM) in a subject by co-transplanting islets with IPTD-MSCs in the subject. In some embodiments, the IPTD-MSCs are expanded and potentiated ex vivo using the methods disclosed above before co-transplanting with the islets to the subject. In some embodiments, the islets are potentiated ex vivo by treating with DMOG. In some embodiments, the islets and the IPTD-MSCs for co-transplantation are harvested simultaneously from a single donor

The following examples are provided to better illustrate the claimed invention and the embodiments described herein, and are not to be interpreted as limiting the scope of the invention. To the extent that specific materials are mentioned, it is merely for purposes of illustration and is not intended to limit the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### Example 1: Intra-Pancreatic Tissue-Derived Mesenchymal Stromal Cells: A Promising Therapeutic Potential with Anti-Inflammatory and Pro-Angiogenic Profiles

As demonstrated in the studies below, IPTD-MSCs were expanded in a GMP-compatible CMRL-1066 medium supplemented with 5% human platelet lysate (hPL). IPTD-MSCs were found to be highly pure, with >95% positive for CD90, CD105, and CD73, and negative for CD45, CD34, CD14 and HLA-DR. Immunofluorescence staining of pancreas tissue demonstrated the presence of CD105⁺ cells in the vicinity of islets. IPTD-MSCs were capable of differentiation into adipocytes, chondrocytes, and osteoblasts in vitro, underscoring their multipotent features. When these cells were cultured in the presence of a low dose of TNF-α, gene expression of tumor necrosis factor alpha-stimulated gene-6 (TSG-6) was significantly increased, compared to control. In contrast, treating cells with dimethyloxallyl glycine (DMOG) (a prolyl 4-hydroxylase inhibitor) enhanced mRNA levels of nuclear factor erythroid 2-related factor 2 (NRF2) and vascular endothelial growth factor (VEGF). A combination of TNF-α and DMOG stimulated the optimal expression of all three genes in IPTD-MSCs. Conditioned medium of IPTD-MSCs treated with a combination of DMOG and TNF-α, contained higher levels of pro-angiogenic (VEGF, IL-6, and IL-8) compared to controls, promoting angiogenesis of human endothelial cells in vitro. In contrast, levels of MCP-1, a pro-inflammatory cytokine, were reduced in conditioned medium of IPTD-MSCs treated with a combination of DMOG and TNF-α.

In this study, MSCs were isolated from the otherwise discarded fractions of pancreatic tissue. These cells, designated as intra-pancreatic tissue-derived (IPTD)-MSCs, were cultured in a GMP-grade and xenoprotein-free culture medium containing human platelet lysate, and conditioned in vitro with TNF-α [27] and DMOG. Changes in gene expression, growth factor and cytokine levels, and angiogenic capacity after conditioning were determined. This study identifies a previously-unappreciated fraction of the pancreatic digest as a useful source of anti-inflammatory and pro-angiogenic MSCs with possible clinical applications.

Materials and Methods

Digestion of Human Pancreata from Cadaveric Donors:

Human cadaveric donor pancreata (n=9) were obtained from an organ procurement organization. Cadaveric donors from which IPTD-MSCs were obtained averaged 33.8±3.1 years of age, 29.8±1.8 body mass index, and 5.1±0.1% hemoglobin A1c (Table 1).

TABLE 1
Characteristics of donors of pancreata used
for islet and IPTD-MSC cell isolation

| Isolation | Age | | | HbA1c | | Cause |
|---|---|---|---|---|---|---|
| number | (yrs) | Race | Sex | (%) | BMI | of death |
| Donor #1 | 47 | C | M | 4.7 | 31.7 | CVA |
| Donor #2 | 27 | H | M | 5.2 | 30.7 | T |
| Donor #3 | 39 | C | M | 5 | 30 | ICB |
| Donor #4 | 17 | H | M | 5.2 | 39.4 | HT |
| Donor #5 | 38 | C | M | 5.2 | 26.3 | HT |
| Donor #6 | 34 | C | M | 4.8 | 33.1 | HT |
| Donor #7 | 27 | H | M | 5 | 24.7 | CVA |
| Donor #8 | 31 | H | M | 5.3 | 31.8 | HT |
| Donor #9 | 44 | AA | M | 5.2 | 20.3 | HT |
| Mean ± | 33.8 ± 3.1 | NA | NA | 5.1 ± 0.1 | 29.8 ± 1.8 | NA |
| SEM | | | | | | |

BMI, body mass index;
HbA1c, hemoglobin A1c;
C, Caucasian;
H, Hispanic;
CVA, Cerebrovascular accident;
HT, Head trauma;
AA, African American;
NA, not applicable

Pancreata from individuals with the criteria of Donation after Cardiac Death and HbA1c>6.5% were excluded from this study. Islet isolation was carried out in a cGMP facility at City of Hope as previously described [28, 29]. Briefly, the pancreas was digested using collagenase supplemented with either thermolysin or neutral protease [28]. The digested pancreatic tissues were collected in eighteen 250-mL conical tubes and centrifuged at 182×g/8° C. for 3 minutes. Pancreatic tissue was collected, washed, and purified in a cold COBE 2991 cell processor (COBE Laboratories Inc., Lakewood, Calif., USA) [30]. Fractions of purified islets were collected and the IPTD-MSCs were cultured as described below.

Intra-Pancreatic Tissue-Derived Cell Harvesting and Culture:

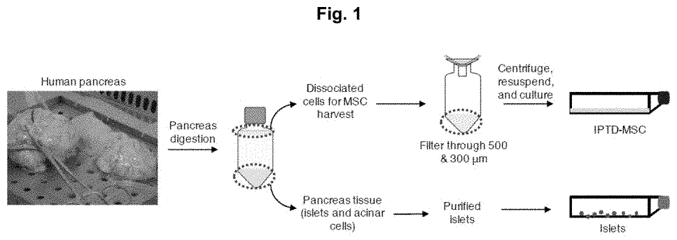

Enzymatic digestion of the whole pancreata released intra-pancreatic tissue and stromal cells. These cells were found to be less dense than the islets and acinar clusters. Under the standard centrifugation condition (182×g for 3 minutes), which was prioritized for islets and acinar clusters, the stromal cells were located at the top layer of the conical tubes (FIG. 1). Until now, this top layer of tissue and cells has been routinely discarded.

To test the hypothesis that IPTD-MSCs can be separated from fractions of the pancreatic tissue, the standard protocol was modified, the upper layer found post-centrifugation was collected and pooled, and the resultant was passed through double layers of mesh filters (500 and 300 μm) to eliminate non-cellular components (FIG. 1). The filtered cells were then washed with CMRL-1066 culture medium and centrifuged at 727×g/8° C. for 3 minutes. The supernatant was aspirated, and the pellet was suspended in CMRL-1066 culture medium supplemented with 5% Human Platelet Lysate (hPL, Compass Biomed, MA) followed by transferring to a 50-mL conical tube. The suspended cells were centrifuged at 727×g/8° C. for 3 minutes. The supernatant was aspirated, and the pellet was suspended in 40 mL of CMRL-1066 medium containing 5% hPL followed by culturing in T-175 adherent flasks (ThermoFisher Scientific, Waltham, Mass.) for 24 hours at 37° C. in 5% CO2 (FIG. 1). Twenty-four hours later, the medium was replaced with fresh CMRL-1066 medium containing 5% hPL. Additional media changes were performed every 48 hours until cells reached ˜80-90% confluence.

Bone Marrow-Derived MSCs:

Bone marrow-derived human MSCs were obtained from healthy individuals as described [31, 32]. All subjects gave written informed consent in accordance with the Declaration of Helsinki. The study protocol was approved by the Medical Ethics Board of Leiden University Medical Center (LUMC).

Characterization of IPTD-MSCs:

Cell morphology. To record the growth and morphology of cultured cells, multiple pictures at different magnifications and time points were obtained using a ckx31 Olympus microscope.

Flow Cytometry.

After reaching 80-90% confluence, cells were dissociated with TrypLE (ThermoFisher, San Diego), washed with DPBS (Corning, Tewksbury, Mass.) twice, and incubated with antibodies specific for cell-surface molecules, including CD90, CD105, CD73, CD9, CD45, CD34, CD14, and HLA-DR (BioLegends, San Diego, Calif.), for 20 minutes at room temperature. In parallel, aliquots of cells were incubated with matched isotype control antibodies from the same supplier. After antibody incubation, cells were washed twice with DPBS and suspended in DPBS for flow cytometry analysis using Sony SA3800 Spectral Analyzer (Sony Biotechnology, San Jose, Calif.). Data analysis was performed using Flowjo software (Tree Star, Ashland, Oreg.). To verify the results, human bone marrow-derived MSCs were cultured in the same medium used for IPTD-MSCs, passaged, and expanded in the same procedures for subsequent analysis.

Immunofluorescent Staining for IPTD-MSCs and Pancreatic Tissue.

Cells were cultured to 70-80% confluence and dissociated into a single-cell suspension using TrypLE as described [33]. Cells and pancreatic tissue were then fixed in 10% cold formalin, prepared in a paraffin block and sectioned. Antigen retrieval was performed using a citric acid-based antigen unmasking solution (Vector, pH 6.0). Sections were treated with protein block (Biogenex, Fremont, Calif.) to reduce background signal, followed by incubation with mouse anti-CD105 antibody (ready to use; Biogenex) and ALEXA 488-conjugated goat anti-mouse IgG antibody (1:200 dilution; ThermoFisher). Guinea pig anti-insulin (ThermoFisher) and ALEXA 647-conjugated goat anti-Guinea pig IgG antibodies (1:200 dilution; ThermoFisher) were used for pancreatic tissue staining only. Fluoroshield™ containing DAPI (Sigma Aldrich St. Louis, Mo.) was used to stain nuclei. Image acquisition was done using an Observer Z1 microscope (Carl Zeiss), with the objective lens set at 20×. Image processing was done using the Zen 2.0 software.

Multilineage Differentiation of IPTD-MSCs.

IPTD-MSCs at the second passage were cultured in T-75 tissue culture flasks until ˜85% confluence. For adipogenic differentiation, IPTD-MSCs were seeded into 6-well plates and cultured in MesenCult™ Adipogenic Differentiation Kit (STEMCELL Technologies, Vancouver, Ca; Cat #05412) for 21 days with media changed every 3 days. The presence of lipid droplets in cells was determined by staining with Oil Red 0 (Sigma, cat #00625) 21 days after culture. For chondrogenic differentiation, IPTD-MSCs were cultured in two 15-mL conical tubes in MesenCult™-ACF Chondrogenic Differentiation medium (STEMCELL Technologies, Cat #05455) for 24 days with media changed every 3 days. After culture, Alcian Blue (Sigma, cat #66011) was used to stain for both fresh cells and the cells fixed in paraffin sections. For osteogenic differentiation, cells were cultured in T-75 tissue culture flask for 22 days with media changed every 3 days. The osteogenic differentiation medium is consisted of CMRL-1066 containing 10 mM β-glycerophosphate (Sigma, Cat #G-6251), 50 μg/mL L-ascorbate acid 2-phosphate (Cayman, Item #16457), 1 μM of dexamethasone (Fresenius Kabi, Cat #401780G), and 3% hPL. Differentiated cells were fixed in paraffin section and stained with von Kossa for calcium deposition. Undifferentiated IPTD-MSCs were cultured in standard culture medium lacking differentiation factors and stained with Oil Red O, Alcian Blue, or von Kossa.

In Vitro Expansion of IPTD-MSCs:

T-175 flasks of ˜80% confluent passage-3 cells were washed twice with DPBS, and 5 ml of TrypLE enzyme was added to the flask. The cells were incubated at 37° C. for 5-10 minutes to dissociate adherent cells, and 10 ml of CMRL-1066 medium was added to terminate enzyme digestion. Cells were collected in 15-ml tubes for centrifugation at 528×g for 3 minutes. The cell pellet was suspended in 5 ml CMRL-1066 with 5% hPL and vortexed. A sample of cells was mixed in a 1:1 ratio with 0.4% trypan blue (ThermoFisher), and from which 20 μL was placed on a counting slide (Cellometer SD100, Nexcelom Bioscience, San Diego, Calif.) and counted using Cellometer Auto T4 (Nexcelom Bioscience, San Diego, Calif.). To further characterize the growth capabilities of these cells, subcultures were performed by placing 5×10⁴ cells in T-25 flasks for 72 hours at 37° C. and 5% CO2. Some cells were grown in CMRL-1066 culture medium alone and others in CMRL-1066 culture medium supplemented with 5% hPL. Culture medium was replaced once during this period. At the end of the culture, cells were dissociated and counted as above. This process was then repeated. After each passage the cell count was multiplied by the dilution factor to calculate the total number of cells per passage.

Cryopreservation of IPTD-MSCs:

Isolated IPTD-MSCs (at passage 3) were cultured to ˜80% confluence, dissociated into single cells with TrypLE, collected and counted. Aliquots of 1×10⁶ cells were divided into cryopreservation tubes, suspended in 10% DMSO in CMRL-1066 medium and stored at −80° C. in a Mr. Frosty Freezing apparatus containing 100% isopropyl alcohol (ThermoFisher). Using this method, IPTD-MSCs were stored for 9 months. The cells were then thawed rapidly in a 37° C. water bath, washed with DPBS and cultured in T-75 tissue culture flasks using CMRL media with 5% hPL. After 48 hours, the cells were noted to be ˜80% confluent and were subjected to subsequent analyses. Viability was assessed with trypan blue.

In Vitro Treatment of IPTD-MSCs with TNF-α and DMOG:

Recombinant human TNF-α protein (R&D Systems, Minneapolis, Minn.) was reconstituted in research-grade water (Hospira, Lake Forest, Ill.) to a concentration of 100 ng/mL, aliquoted and stored at −20° C. Dimethyloxallyl glycine (DMOG; Cayman Chemicals, Ann Arbor, Mich.) was dissolved in water to yield a stock solution of 57.1 mM, aliquoted (100 μL) and stored at −80° C. IPTD-MSCs were incubated in T-25 flasks in 5 ml of CMRL-1066 medium supplemented with 5% hPL until ˜50% confluent. Cells were cultured for 24 hours in CMRL 1066 medium, or medium containing 10 ng/ml TNF-α, 1 mM DMOG or 1 mM DMOG and 10 ng/ml TNF-α. Following treatment, cells were collected in 1.7-ml Eppendorf tubes and stored in RLT buffer (Qiagen, Germantown, Md.) at −80° C. for future preparation of cDNA.

Quantitative Real-Time PCR:

The TaqMan Gene Expression Assay system (ThermoFisher Scientific) was used to quantify β-ACTIN, TSG-6, NRF2, and VEGF mRNA levels. Total RNA was extracted using a Qiagen Mini Kit (Cat. No. 51306) and converted into cDNA. Real-time quantitative PCR was run in duplicate on a ViiA™ 7 Real-Time PCR System with a 384-well block (ThermoFisher Scientific). Thermal cycles were programmed for 20 seconds at 95° C. for the initial denaturation, followed by 45 cycles of 120 seconds at 95° C. for denaturation, 30 seconds at 60° C. for annealing, 60 seconds at 72° C. for extension, and a final extension at 72° C. for 10 minutes. All PCR runs were performed with negative (water) and positive controls. β-ACTIN was used as an internal control to quantify relative gene expression.

Cytokine Assay:

Supernatants from cells cultured for 24 hours under various conditions (medium alone, or medium plus DMOG, TNF-α, or DMOG+TNF-α) were collected, and cytokine analysis performed using a Luminex assay kit (Bio-Rad, Hercules, Calif.) according to the manufacturer's protocol. The following growth factors/cytokines were measured: VEGF, IL-6, IL-8, MMP-9, MCP-1, MMP-2, IL-4, IL-10 and IL-113. Sample were measured in duplicate.

In Vitro Angiogenesis Assay:

Angiogenic capacity was assessed by quantifying endothelial tube formation [34]. Human umbilical vein endothelial cells (HUVECs) (Cell Applications Inc., San Diego, Calif.; Cat #200p-05n) between passages 2 to 6 were cultured in a standard medium. Cells (1×10⁵ cells/well) were plated in 24-well plates (Fisher, Cat #930186) coated with Matrigel (Corning, Cat #356234), and incubated for 30 minutes to allow cell attachment. Supernatants (150 μL/well) from control and stimulated IPTD-MSCs (DMOG, TNF-α, or DMOG+TNF-α) were added to obtain a total volume of 300 μL per well. Plates were then incubated at 37° C., 5% CO₂ for 24 hours. At 4 and 24 hours, the wells were visualized using a Leica microscope and representative photographic images were obtained. Total endothelial tube number and tube length were determined using ImageJ software (NIH, Bethesda, Md.).

Statistical Analysis:

Data was analyzed with GraphPad Prism software (GraphPad Software 8.0, La Jolla, Calif.). ANOVA one-way analysis of variance was used to compare multiple experimental groups followed by the Tukey multiple comparisons test to compare the mean values between any two groups. All the values were expressed as mean±standard error of mean (SEM). For all the tests, p<0.05 was considered significant.

Results

A Chemically-Defined Medium Supports the Growth IPTD-MSCs.

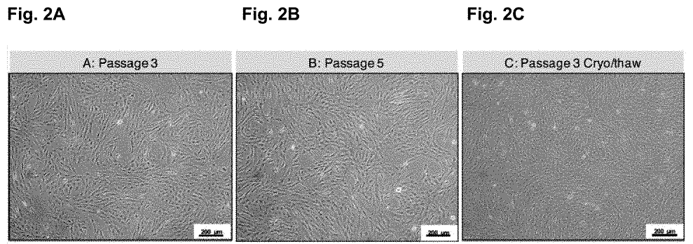

To develop a GMP-compatible culture medium, whether hPL could support the growth of IPTD-MSCs in the absence of fetal bovine serum was tested. Under phase-contrast microscopic evaluation, IPTD-MSCs displayed elongated and spindle shapes (FIG. 2A), a morphology consistent with the classic MSCs derived from other tissues [35]. Next, IPTD-MSCs were dissociated and replated multiple times and it was found that the spindle shape morphology was preserved throughout numerous passages (FIG. 2B). IPTD-MSCs were cryopreserved for 9 months, thawed and cultured, and again the cell morphology remained stable (FIG. 2C).

hPL is Required for the Growth of IPTD-MSCs.

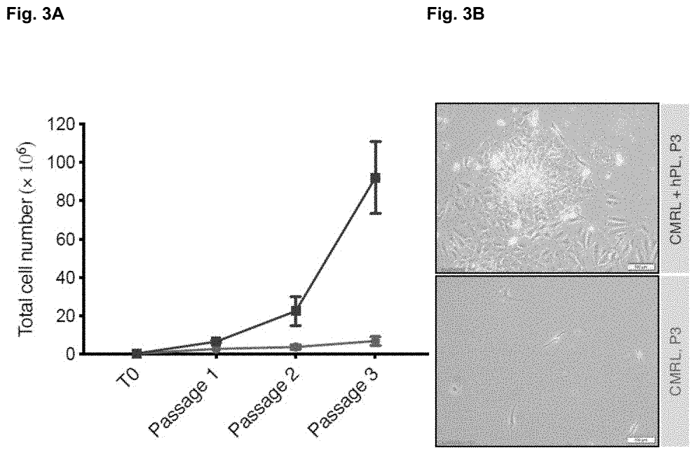

Whether hPL is essential for the growth of IPTD-MSCs was tested by removing it from the culture medium and passaging cells for three generations. IPTD-MSCs grown in hPL-repleted medium expanded an average of 10.8-fold, while those cultured in medium lacking hPL showed minimal to no expansion (FIG. 3), suggesting that hPL is required for the expansion of IPTD-MSCs in vitro.

IPTD-MSCs Display Classic MSC Cell-Surface Markers.

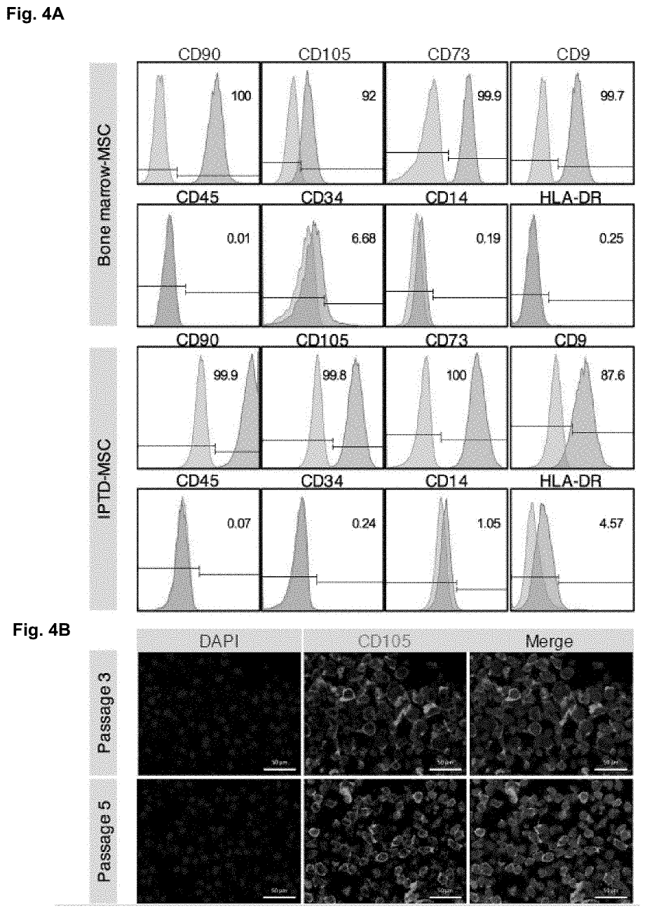

To test whether IPTD-MSCs express known markers found on MSCs isolated from bone marrow and other organs [36], flow cytometry analysis was performed. Cell-surface marker expression was confirmed using bone marrow-derived MSCs. As expected, the majority of bone marrow-derived MSCs expressed CD90, CD105, CD73 and CD9, and showed minimal to no expression of CD45 (pan-leukocytes), CD34 (hematopoietic cells), CD14 (macrophages) and HLA-DR (antigen presenting cells) (FIG. 4A).

IPTD-MSCs were passaged 3 times, dissociated into a single-cell suspension and stained with the above-mentioned antibodies. Compared to isotype-control staining, the vast majority of IPTD-MSCs stained positive for CD90 (99.2±0.3%), CD105 (99.8±0.2%), CD73 (99.6±0.3%), and CD9 (86.8±2.6%) (FIG. 4A). Minimal expression of CD45 (0.3±0.2%), CD34 (0.3±0.0%), CD14 (1.5±0.8%), and HLA-DR was found (FIG. 4A). Expression of CD105 on the cell surface of IPTD-MSCs at passages 3 and 5 was further visualized using immunofluorescent staining (FIG. 4B). Taken together, IPTD-MSCs expressed classic positive and lacked negative markers for MSCs, suggesting that they reside within the MSC family of cells.

CD105⁺ Cells Localize in the Pancreas Near Insulin-Expressing Cells.

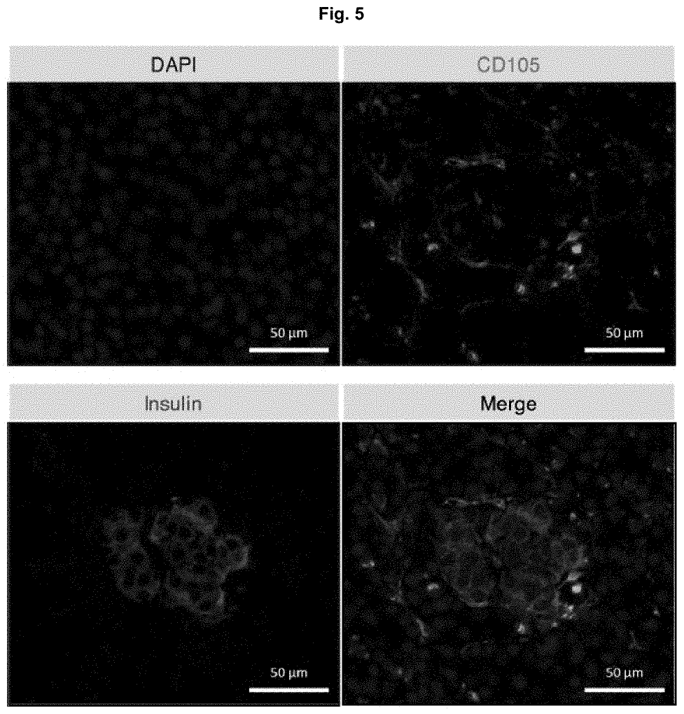

To rule out the possibility that the ex vivo IPTD-MSC growth and expansion was due to in-vitro selection or artifact, whether CD105⁺ cells were present in the endogenous pancreas was examined. Pancreatic tissue sections were co-stained with CD105 and insulin. CD105⁺ cells were detected in the pancreatic tissue and were located adjacent to the insulin-expressing islets (FIG. 5). This result confirms the existence of CD105⁺ cells in the adult human pancreas.

IPTD-MSCs have Potentials to Differentiate into Multiple Cell Lineages In Vitro.

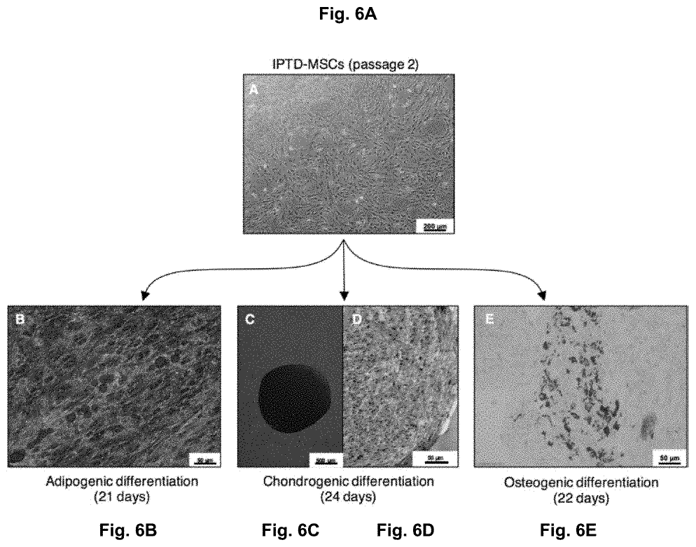

A typical feature of MSCs is the ability to assume lineage-specific cell phenotypes after exposure to certain growth factors. IPTD-MSCs were exposed to adipogenic, chondrogenic and osteogenic growth conditions. Under these differentiation conditions, IPTD-MSCs were found to give rise to the appropriate lineage-associated phenotypes, including cells positively-stained for Oil Red O (adipocytes), Alcian Blue (chondrocytes) or von Kossa (osteoblasts) (FIG. 6). In contrast, undifferentiated IPTD-MSCs showed no lineage-specific staining.

TNF-α and DMOG Upregulate Immune-Regulatory and Angiogenic Genes in IPTD-MSCs.

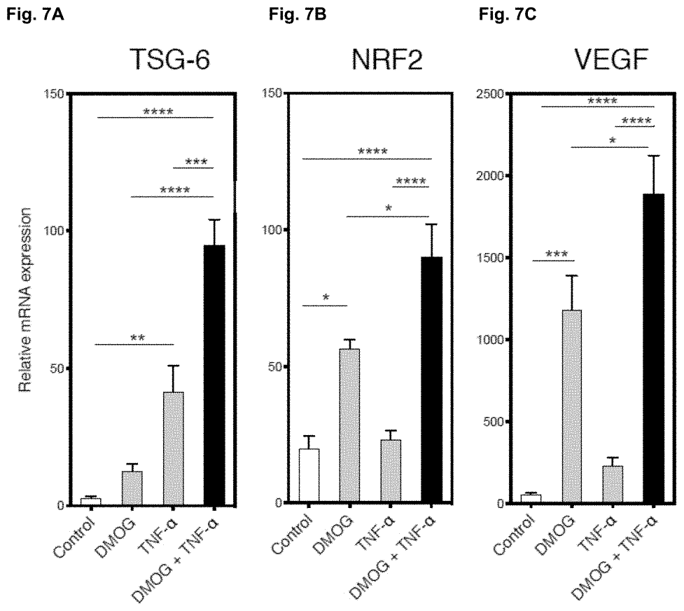

Next, whether IPTD-MSCs were amenable to in vitro conditioning was tested. IPTD-MSCs were stimulated with TNF-α and/or DMOG, and the expression of known immune-modulating and angiogenic genes was determined. Consistent to previous findings [27], TSG-6 gene expression levels were significantly increased in cells treated with 10 ng/ml TNF-α (p<0.01) but not DMOG alone (FIG. 7A), compared to control. Addition of DMOG to TNF-α further increased the expression of TSG-6 (p<0.0001) (FIG. 7A). NRF2 and VEGF expression were significantly increased when cells were treated with DMOG (p<0.05 and p<0.001 respectively, FIGS. 7B, 7C) but not TNF-α alone, compared to control. Addition of DMOG to TNF-α further enhanced the expression of NRF2 and VEGF (p<0.0001, FIG. 7B, C). These results suggest that, while TNF-α and DMOG display divergent effects, the combination of the two best enhances in IPTD-MSCs genes that are known to modulate immune responses and angiogenesis.

TNF-α and DMOG Alter Growth Factors and Cytokines Released by IPTD-MSCs.

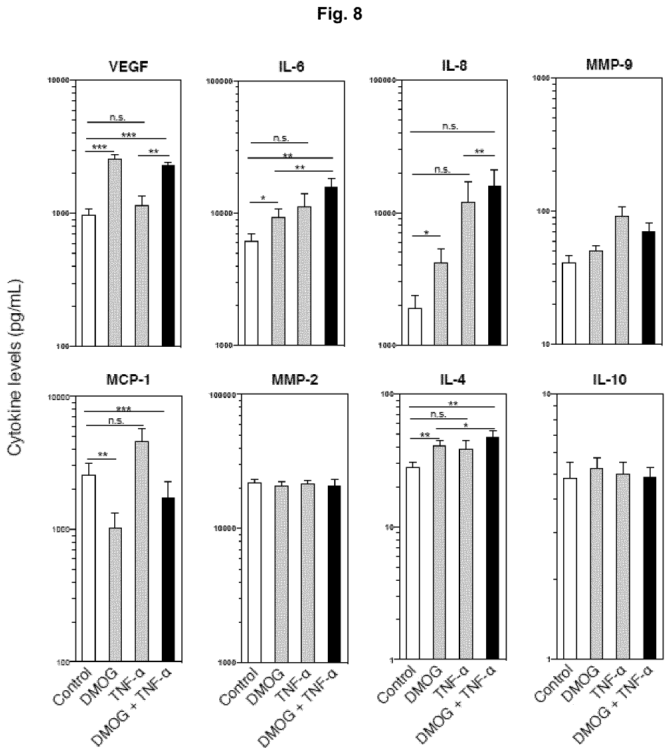

To further characterize IPTD-MSCs, proteins released from these cells were examined. IPTD-MSCs were cultured for 24 hours in the presence of exogenous TNF-α, DMOG or both, and the resulting culture media were examined by Luminex assay. Compared to control, stimulation of IPTD-MSCs with DMOG alone enhanced the secretion of VEGF, IL-6, IL-8, and IL-4 (FIG. 8; comparing the 1st to the 2nd bars). Stimulation of IPTD-MSCs with the combination of DMOG plus TNF-α enhanced the secretion of VEGF, IL-6, and IL-4 (FIG. 8; comparing the 1st to the 4th bars). While the stimulation of IPTD-MSCs with TNF-α alone did not have an effect on any of the cytokines examined compared to controls. The addition of TNF-α to DMOG enhanced the secretion of IL-6 and IL-4 (FIG. 8; comparing the 2nd to the 4th bars). Levels of MCP-1 were reduced in the conditioned media of IPTD-MSCs treated with DMOG or DMOG plus TNF-α, but not with TNF-α alone. Levels of MMP-9, MMP-2 and IL-10 were not changed in response to various conditioning while L-1β was undetectable.

Conditioned Medium from IPTD-MSCs Stimulated with DMOG Promotes Angiogenic Activity of Endothelial Cells.

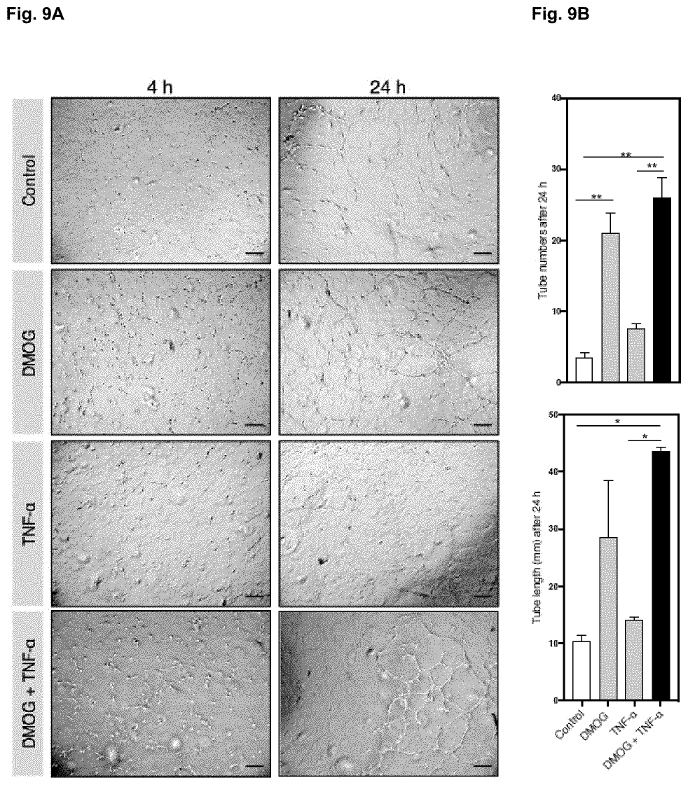

Endothelial-cell tube formation is an acknowledged angiogenic metric indicative of cell migration, adhesion and re-organization. To test this, HUVECs were exposed to conditioned media from IPTD-MSCs stimulated with TNF-α, DMOG or both. Four hours post-plating, HUVECs treated with various IPTD-MSC conditioned media displayed similar morphology without significant difference in tube number or length, regardless of the source of the media. By 24 hours, endothelial tube formation was apparent (FIG. 9A). HUVECs incubated with media from IPTD-MSCs treated with DMOG (21.0±2.0, p<0.01) or DMOG+TNF-α (26.0±2.0, p<0.01) displayed increased numbers of cell tubes, compared to control (3.5±0.5) (FIG. 9B). Similarly, tube length was significantly higher in HUVECs exposed to IPTD-MSC conditioned media stimulated with DMOG (28.6±9.9 mm, p<0.05) or DMOG+TNF-α (43.6±0.8 mm, p<0.05), compared to control (10.3±1.0 mm) (FIG. 9B). TNF-α by itself had no effect on tube number or length, and TNF-α did not enhance the effects of DMOG (FIG. 9B; comparing the 2nd and the 4th bars), suggesting that DMOG is the sole stimulant to enhance IPTD-MSC-mediated angiogenesis in vitro.

Discussion

As disclosed herein, an MSC population that resides within pancreatic tissues is identified, which can be separated during islet isolation. These cells are named intra-pancreatic tissue-derived (IPTD)-MSCs, in agreement with the recent call for nomenclature of MSCs in relation to their tissue of origin [37-39]. In culture, IPTD-MSCs displayed features similar to classic bone marrow- or umbilical cord blood-derived MSCs, including adherence to culture-grade plastic surfaces, spindle-shaped morphology, expression of appropriate surface markers (positive for CD90, CD105, and CD73, and negative for CD45, CD34, CD14 and HLA-DR), and capacity for proliferation and multilineage differentiation. Furthermore, when IPTD-MSCs were treated with a combination of TNF-α and DMOG, the mRNA levels of TSG-6, NRF2, and VEGF were increased, secretion from IPTD-MSCs of VEGF, IL-6, IL-8, and IL-4 was increased, secretion of MCP-1 was decreased, and endothelial cell tube formation was enhanced. Together, these results suggest IPTD-MSCs conditioned by TNF-α and DMOG have anti-inflammatory and pro-angiogenic potential.

The cell population, isolation and culture method of IPTD-MSCs described herein have both differences and similarities over other previously-published MSCs [40]. In this study, cells were isolated from intra-pancreatic tissue as a part of islet isolation procedure from a single donor. IPTD-MSCs were harvested from an otherwise discarded component after routine pancreatic digestion and islet isolation. The GMP-compatible protocol used for culturing these cells led to the production of large numbers of highly-purified MSCs. CMRL-1066 was selected as the base medium to propagate IPTD-MSCs because CMRL-1066 is routinely used to culture islets for transplantation, thus reducing the burden for future clinical translation. Additionally, animal products in culture media was eliminated by using hPL, which will lower the risks of infection, allergic reactions and product variability. Similar to MSCs derived from other tissue sources, IPTD-MSCs are capable of differentiation into adipocyte, chondrocyte and osteoblast linages, demonstrating the multi-lineage potential of IPTD-MSCs.

Disclosed herein is an approach that allows for harvesting islets and IPTD-MSCs simultaneously from a single donor under GMP conditions, facilitating direct clinical application. Harvesting IPTD-MSCs during human islet isolation makes quality evaluation of isolated cells rapid and reliable, and suggests opportunities for immediate clinical applications. Previously, autologous bone marrow-derived MSCs have been used simultaneously in living-related kidney transplant recipients [41]. Moreover, bone marrow-derived MSCs were expanded in the same medium of CMRL-1066 supplemented with hPL, and it was found that these MSCs where similar in phenotype and characteristics compared to IPTD-MSCs, suggesting that the medium could be used to isolate MSCs from other tissue sources. IPTD-MSC culture medium used in this study is xeno-protein free and cGMP-compatible. The isolated IPTD-MSCs were expandable and can be produced in large scale using this culture medium. Conventionally, fetal bovine serum is supplemented in selected culture media to promote the growth of MSCs from different tissue sources [42]. However, the use of non-human serum to culture cells carries the potential of transmitting infectious agents [43], immunizing effects [44], and lot-to-lot variability. In this regard, human platelet lysate has been used to replace fetal bovine serum for clinical-scale MSC expansion [45]. In these studies, hPL was supplemented in minimal essential medium (MEM) to culture MSCs. In the current study, hPL was used to supplement the CMRL-1066 that has been optimized for human islets culture, and the culture system employed herein allows for optimum survival of IPTD-MSCs. This is important since a single medium system can be used for both cell sources to facilitate co-transplantation of islets and IPTD-MSCs in future studies.

This disclosure also highlights the benefit of harvesting multiple cell types from tissue fractions of a single donor organ as part of the islet isolation procedure. It is conceivable that immunophenotypic characterization and identification of additional novel cell types reside within this tissue fraction would be valuable to study pancreatic pathophysiology arising from various diseases.

MSCs are known to reduce inflammation and enhance healing, and these functions can be further manipulated ex vivo to enhance capacities for cell therapies. Compared to control, IPTD-MSCs exposed to a combination of TNF-α and DMOG, compared to single reagents, exerted a better overall outcome. Except for TSG-6 expression, no other molecules, including the secreted factors examined in this study, were affected by TNF-α treatment alone. In contrast, DMOG alone was able to induce NRF2, VEGF expression, as well as the secretion of VEGF, IL-6, IL-8, and IL-4. These results demonstrate a dominant effect of DMOG over TNF-α. However, TNF-α was able to augment the effects brought by DMOG in increasing the expression of TSG-6, NRF2, and VEGF, and enhancing secretion from IPTD-MSCs of IL-6, IL-8 and IL-4. Regardless, the combination of TNF-α and DMOG appeared to be optimal for the examined outcomes, including the expression of TSG-6, NRF2, and VEGF, secretion of VEGF, IL-6, IL-8, and IL-4, and endothelial tube formation. This disclosure shows a beneficial effect on MSCs by conditioning with the combination of TNF-α and DMOG.

IL-4 levels were significantly increased by the combination of DMOG and TNF-α as compared to the control or DMOG alone, whereas IL-10 production was unchanged. This is in line with previous reports demonstrating that MSCs do not secrete IL-10, but stimulate other immune cells to secrete this cytokine [46]. MCP-1 (monocyte chemoattractant protein-1) is often increased upon treatment with inflammatory cytokines. The treatment of IPTD-MSCs with DMOG and TNF-α led to a reduction of MCP-1. Taken together, these results show the production of anti-inflammatory molecules in IPTD-MSCs.

VEGF, IL-6, IL-8, and MMP-9 are known pro-angiogenic factors [47-51], which may be responsible for the observed enhancement of endothelial cell tube formation. Upregulation and secretion of pro-angiogenic factors are important for several reasons: i) MSCs from individuals with diabetes showed lowered angiogenic capacity [52] than those from individuals without diabetes, although another study reported that MSCs isolated from the bone marrow of T1D donors were phenotypically and functionally similar to those isolated from healthy individuals [53]; ii) treatment of islets with the iron chelator deferoxamine stabilized HIF-α and enhanced islet VEGF levels [54]; and iii) treatment with exogenous VEGF improves islet engraftment [55] and β-cell mass [56], in part through increased angiogenesis. Islet survival and function post-transplantation are adversely impacted by hypoxia [57]. Thus, processes that render islets hypoxia-resistant, such as increasing VEGF expression and secretion, should have beneficial effects in islet transplantation. The fact that the combination of DMOG and TNF-α also enhances TSG-6, NRF2, and VEGF gene expression from bone-marrow derived MSCs highlights the potential use of DMOG and TNF-α to condition MSCs other than IPTD-MSCs. Further, the upregulation of TSG-6 in both IPTD-MSCs and bone marrow-derived MSCs underscores the concept of employing TSG-6 as a marker of anti-inflammatory capacity [58].

The proximity of MSCs to islets within the pancreatic tissue, indicates a possible role for these MSCs in protecting islets from metabolic stress and inflammation.

The mechanism by which MSCs protect human islets include the expression of anti-inflammatory and pro-angiogenic genes [8, 9]. Tumor necrosis factor alpha-stimulated gene-6 (TSG-6) induced by TNF-α has anti-inflammatory properties [10-12]. Nuclear factor erythroid 2-related factor 2 (NRF2) is important in enhancing islet graft survival and function [13, 14]. Additionally, dimethyloxallyl glycine (DMOG), which targets prolyl-4-hydroxylase to prevent the degradation of hypoxia-inducible factor-1a [15] and upregulate vascular endothelial growth factor (VEGF) [16], could be a possible conditioning factor for improving MSC function. This mechanism is not limited to MSCs isolated from the pancreas, nor is the protective effect of MSCs limited to the protection of islets. Thus, the conditioning protocols described in the embodiments above may apply to MSCs isolated from any donor organ or tissue, and the conditioned MSCs may be used in treatments for any suitable therapeutic application.

MSCs have been isolated from various sites including subcutaneous adipose tissue [17, 18], bone marrow [19, 20], skeletal muscle [21], umbilical cord blood [22], ocular limbus [23], and amniotic fluid [24]. Blood- and adipose-derived MSCs are widely investigated due to their accessibility, expandability, differentiability, and clinical applicability [25, 26]. During the enzymatic digestion of the cadaveric pancreas, cells are liberated, together with islets, which can then be separated and characterized.

### Example 2: Stabilization of Hypoxia Inducible Factor-1α (HIF-1α) Improves Human Islet Survival and Function

The hypoxia-inducible factor-1α (HIF-1α) pathway is upregulated in response to a drop in oxygen. At the cellular level, protection from rapid changes in oxygen occurs through suppression of prolyl hydroxylase (PHD)-mediated degradation of constitutively produced HIF-1α. As HIF-1 levels increase down-stream target genes are activated affording protection from hypoxia. Conversely, as oxygen levels are restored, PHD is de-repressed lowering HIF-1α levels. Transplantation of cells, tissues and organs invariably results in varying degrees of hypoxic and ischemic injury. This has been found to occur in clinical islet transplantation for type 1 diabetes, and results in loss of transplant cell mass and decreased acute and long-term cell function. The hypothesis that PHD suppression would upregulate key survival gene pathways to improved islet response to stress and hypoxia was tested.

Islets of high and low purity, and mesenchymal stem cells (MSCs) as a potential source of human islets, isolated from human donor pancreata were treated with the PHD inhibitor DMOG. Alterations in gene expression, cell proliferation, survival and metabolism were characterized.

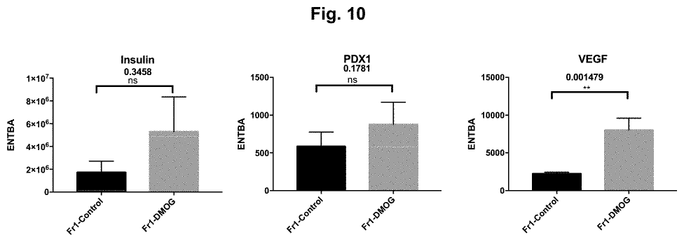

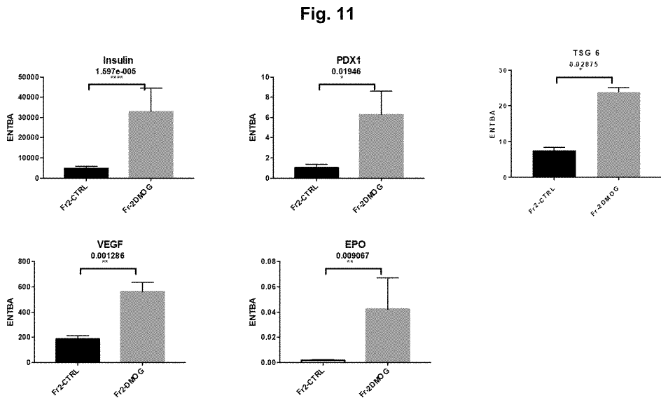

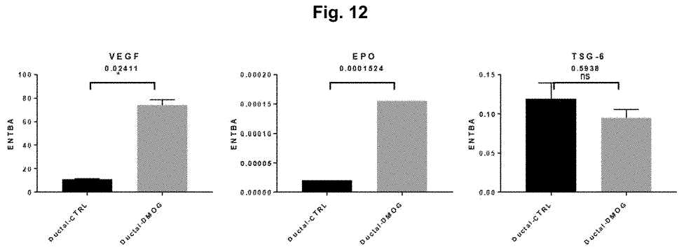

As shown in FIGS. 10-12, pancreatic-derived human islets and MSCs were treated with DMOG or control at several concentrations and time intervals. The levels of HIF-1a were increased and PHD decreased in DMOG-treated versus control cells, and in a dose-dependent manner. Concurrently, DMOG treatment resulted in upregulation of key survival genes including erythropoietin (EPO) and other proteins, including glycolytic enzyme, glucose transporter-1 (Glut-1) and vascular endothelial growth factor (VEGF). Functionally, treated islets and MSCs were found less susceptible to cytokine injury and had a higher proliferative capacity in vitro. Finally, treated islets showed increased engraftment in diabetic mice and this was associated with improved glucose homeostasis.

Suppression of PHD renders human pancreatic-derived islets and MSCs less sensitive to inflammatory stress and more proliferative, while protecting islets from transplantation-mediated loss and failure.

These results point to a potential benefit from DMOG treatment of islets in clinical transplantation to improve graft take and function. They further suggest this benefit extends to MSCs as a source of islets. While not tested, it will be interesting if backing up the treatment time to capture pancreata prior to and/or during harvest and transportation further improves islet engraftment and function.

### REFERENCES

1 Roberts I. Mesenchymal stem cells. Vox Sang 2004; 87 Suppl 2:38-41.
2 Olsen T R, Ng K S, Lock L T et al. Peak MSC—Are We There Yet? Front Med (Lausanne) 2018; 5:178.
3 Abdi R, Fiorina P, Adra C N et al. Immunomodulation by mesenchymal stem cells: a potential therapeutic strategy for type 1 diabetes. Diabetes 2008; 57:1759-1767.
4 Guan L X, Guan H, Li H B et al. Therapeutic efficacy of umbilical cord-derived mesenchymal stem cells in patients with type 2 diabetes. Exp Ther Med 2015; 9:1623-1630.
5 Liu X, Zheng P, Wang X et al. A preliminary evaluation of efficacy and safety of Wharton's jelly mesenchymal stem cell transplantation in patients with type 2 diabetes mellitus. Stem Cell Res Ther 2014; 5:57.
6 Carlsson P O, Schwarcz E, Korsgren O et al. Preserved beta-cell function in type 1 diabetes by mesenchymal stromal cells. Diabetes 2015; 64:587-592.
7 Berman D M, Willman M A, Han D et al. Mesenchymal stem cells enhance allogeneic islet engraftment in nonhuman primates. Diabetes 2010; 59:2558-2568.
8 Yeung T Y, Seeberger K L, Kin T et al. Human mesenchymal stem cells protect human islets from pro-inflammatory cytokines. PLoS One 2012; 7:e38189.
9 Brandhorst D, Brandhorst H, Acreman S et al. Hypoxia-Induced Damage in Human Islets Is Reduced With the Use of Mesenchymal Stem Cell-Preconditioned Medium. Transplant Proc 2017; 49:2330-2332.
10 Lee R H, Pulin A A, Seo M J et al. Intravenous hMSCs improve myocardial infarction in mice because cells embolized in lung are activated to secrete the anti-inflammatory protein TSG-6. Cell Stem Cell 2009; 5:54-63.
11 Prockop D J. Inflammation, fibrosis, and modulation of the process by mesenchymal stem/stromal cells. Matrix Biol 2016; 51:7-13.
12 Oh J Y, Roddy G W, Choi H et al. Anti-inflammatory protein TSG-6 reduces inflammatory damage to the cornea following chemical and mechanical injury. Proc Natl Acad Sci USA 2010; 107:16875-16880.
13 Li S, Vaziri N D, Masuda Y et al. Pharmacological activation of Nrf2 pathway improves pancreatic islet isolation and transplantation. Cell Transplant 2015; 24:2273-2283.
14 Yagishita Y, Uruno A, Chartoumpekis D V et al. Nrf2 represses the onset of type 1 diabetes in non-obese diabetic mice. J Endocrinol 2019.
15 Milkiewicz M, Pugh C W, Egginton S. Inhibition of endogenous HIF inactivation induces angiogenesis in ischaemic skeletal muscles of mice. J Physiol 2004; 560:21-26.
16 Zhang J, Guan J, Qi X et al. Dimethyloxaloylglycine Promotes the Angiogenic Activity of Mesenchymal Stem Cells Derived from iPSCs via Activation of the PI3K/Akt Pathway for Bone Regeneration. Int J Biol Sci 2016; 12:639-652.
17 Zuk P A, Zhu M, Mizuno H et al. Multilineage cells from human adipose tissue: implications for cell-based therapies. Tissue Eng 2001; 7:211-228.
18 Arzouni A A, Vargas-Seymour A, Rackham C L et al. Mesenchymal stromal cells improve human islet function through released products and extracellular matrix. Clin Sci (Lond) 2017; 131:2835-2845.
19 Lavoie J R, Creskey M M, Muradia G et al. Brief Report: Elastin Microfibril Interface 1 and Integrin-Linked Protein Kinase Are Novel Markers of Islet Regenerative Function in Human Multipotent Mesenchymal Stromal Cells. Stem Cells 2016; 34:2249-2255.
20 Kayleigh M. Van Megen E-JvtW, Julia Lages Motta, Bernice Dekker, Tatjana Nikolic, Bart O. Roep. Activated mesenchymal stromal cells process and present antigens regulating adaptive immunity. Front Immunol;| doi: 10.3389/fimmu.2019.00694 (in press).
21 Williams J T, Southerland S S, Souza J et al. Cells isolated from adult human skeletal muscle capable of differentiating into multiple mesodermal phenotypes. Am Surg 1999; 65:22-26.
22 Wang Y H, Yang Z Q, Zhu S F et al. Comparative study of methotrexate and human umbilical cord mesenchymal stem cell transplantation in the treatment of rheumatoid arthritis. J Biol Regul Homeost Agents 2018; 32:599-605.
23 Holan V, Trosan P, Cejka C et al. A Comparative Study of the Therapeutic Potential of Mesenchymal Stem Cells and Limbal Epithelial Stem Cells for Ocular Surface Reconstruction. Stem Cells Transl Med 2015; 4:1052-1063.
24 Roubelakis M G, Bitsika V, Zagoura D et al. In vitro and in vivo properties of distinct populations of amniotic fluid mesenchymal progenitor cells. J Cell Mol Med 2011; 15:1896-1913.
25 Zuk P A, Zhu M, Ashjian P et al. Human adipose tissue is a source of multipotent stem cells. Mol Biol Cell 2002; 13:4279-4295.
26 Horwitz E M, Gordon P L, Koo W K et al. Isolated allogeneic bone marrow-derived mesenchymal cells engraft and stimulate growth in children with osteogenesis imperfecta: Implications for cell therapy of bone. Proc Natl Acad Sci USA 2002; 99:8932-8937.
27 Lee R H, Yu J M, Foskett A M et al. TSG-6 as a biomarker to predict efficacy of human mesenchymal stem/progenitor cells (hMSCs) in modulating sterile inflammation in vivo. Proc Natl Acad Sci USA 2014; 111:16766-16771.
28 Qi M, McFadden B, Valiente L et al. Human Pancreatic Islets Isolated From Donors With Elevated HbA1c Levels: Islet Yield and Graft Efficacy. Cell Transplant 2015; 24:1879-1886.
29 Khiatah B, Tucker A, Chen K T et al. Evaluation of collagenase gold plus BP protease in isolating islets from human pancreata. Islets 2018:e1417716.
30 Qi M, Valiente L, McFadden B et al. The Choice of Enzyme for Human Pancreas Digestion is a Critical Factor for Increasing the Success of Islet Isolation. Transplant Direct 2015; 1.
31 van der Garde M, van Pel M, Millan Rivero J E et al. Direct Comparison of Wharton's Jelly and Bone Marrow-Derived Mesenchymal Stromal Cells to Enhance Engraftment of Cord Blood CD34(+) Transplants. Stem Cells Dev 2015; 24:2649-2659.
32 van Megen K M, van't Wout E T, Lages Motta J et al. Activated Mesenchymal Stromal Cells Process and Present Antigens Regulating Adaptive Immunity. Front Immunol 2019; 10:694.
33 Iglesias I, Valiente L, Shiang K D et al. The effects of digestion enzymes on islet viability and cellular composition. Cell transplantation 2012; 21:649-655.
34 Chen Z, Lai T C, Jan Y H et al. Hypoxia-responsive miRNAs target argonaute 1 to promote angiogenesis. J Clin Invest 2013; 123:1057-1067.
35 Wagner W, Wein F, Seckinger A et al. Comparative characteristics of mesenchymal stem cells from human bone marrow, adipose tissue, and umbilical cord blood. Exp Hematol 2005; 33:1402-1416.
36 Arzouni A A, Vargas-Seymour A, Nardi N et al. Using Mesenchymal Stromal Cells in Islet Transplantation. Stem Cells Transl Med 2018.
37 Sipp D, Robey P G, Turner L. Clear up this stem-cell mess. Nature 2018; 561:455-457.
38 Path G, Perakakis N, Mantzoros C S et al. Stem cells in the treatment of diabetes mellitus—focus on mesenchymal stem cells. Metabolism 2018.
39 Zazzeroni L, Lanzoni G, Pasquinelli G et al. Considerations on the harvesting site and donor derivation for mesenchymal stem cells-based strategies for diabetes. CellR4 Repair Replace Regen Reprogram 2017; 5.
40 Lo Furno D, Mannino G, Cardile V et al. Potential Therapeutic Applications of Adipose-Derived Mesenchymal Stem Cells. Stem Cells Dev 2016.
41 Tan J, Wu W, Xu X et al. Induction therapy with autologous mesenchymal stem cells in living-related kidney transplants: a randomized controlled trial. JAMA 2012; 307:1169-1177.
42 Heiskanen A, Satomaa T, Tiitinen S et al. N-glycolylneuraminic acid xenoantigen contamination of human embryonic and mesenchymal stem cells is substantially reversible. Stem Cells 2007; 25:197-202.
43 Sundin M, Ringden O, Sundberg B et al. No alloantibodies against mesenchymal stromal cells, but presence of anti-fetal calf serum antibodies, after transplantation in allogeneic hematopoietic stem cell recipients. Haematologica 2007; 92:1208-1215.
44 Reinisch A, Bartmann C, Rohde E et al. Humanized system to propagate cord blood-derived multipotent mesenchymal stromal cells for clinical application. Regen Med 2007; 2:371-382.
45 Schallmoser K, Bartmann C, Rohde E et al. Human platelet lysate can replace fetal bovine serum for clinical-scale expansion of functional mesenchymal stromal cells. Transfusion (Paris) 2007; 47:1436-1446.
46 Najar M, Raicevic G, Crompot E et al. The Immunomodulatory Potential of Mesenchymal Stromal Cells: A Story of a Regulatory Network. J Immunother 2016; 39:45-59.
47 Romieu-Mourez R, Francois M, Boivin M N et al. Cytokine modulation of TLR expression and activation in mesenchymal stromal cells leads to a proinflammatory phenotype. J Immunol 2009; 182:7963-7973.
48 Webb A H, Gao B T, Goldsmith Z K et al. Inhibition of MMP-2 and MMP-9 decreases cellular migration, and angiogenesis in in vitro models of retinoblastoma. BMC Cancer 2017; 17:434.
49 Cohen T, Nahari D, Cerem L W et al. Interleukin 6 induces the expression of vascular endothelial growth factor. J Biol Chem 1996; 271:736-741.
50 Gopinathan G, Milagre C, Pearce O M et al. Interleukin-6 Stimulates Defective Angiogenesis. Cancer Res 2015; 75:3098-3107.
51 Li A, Dubey S, Varney M L et al. IL-8 directly enhanced endothelial cell survival, proliferation, and matrix metalloproteinases production and regulated angiogenesis. J Immunol 2003; 170:3369-3376.
52 Kim H, Han J W, Lee J Y et al. Diabetic Mesenchymal Stem Cells Are Ineffective for Improving Limb Ischemia Due to Their Impaired Angiogenic Capability. Cell Transplant 2015; 24:1571-1584.
53 Davies L C, Alm J J, Heldring N et al. Type 1 Diabetes Mellitus Donor Mesenchymal Stromal Cells Exhibit Comparable Potency to Healthy Controls In Vitro. Stem Cells Transl Med 2016; 5:1485-1495.
54 Bradley B, Prowse S J, Bauling P et al. Desferrioxamine treatment prevents chronic islet allograft damage. Diabetes 1986; 35:550-555.
55 Langlois A, Bietiger W, Seyfritz E et al. Improvement of rat islet viability during transplantation: validation of pharmacological approach to induce VEGF overexpression. Cell Transplant 2011; 20:1333-1342.
56 Brissova M, Aamodt K, Brahmachary P et al. Islet microenvironment, modulated by vascular endothelial growth factor-A signaling, promotes beta cell regeneration. Cell Metab 2014; 19:498-511.
57 Fraker C A, Cechin S, Alvarez-Cubela S et al. A physiological pattern of oxygenation using perfluorocarbon-based culture devices maximizes pancreatic islet viability and enhances beta-cell function. Cell Transplant 2013; 22:1723-1733.
58 Prockop D J. The exciting prospects of new therapies with mesenchymal stromal cells. Cytotherapy 2017; 19:1-8.
59 Garcia-Contreras M, Brooks R W, Boccuzzi L et al. Exosomes as biomarkers and therapeutic tools for type 1 diabetes mellitus. Eur Rev Med Pharmacol Sci 2017; 21:2940-2956.
60 Shigemoto-Kuroda T, Oh J Y, Kim D K et al. MSC-derived Extracellular Vesicles Attenuate Immune Responses in Two Autoimmune Murine Models: Type 1 Diabetes and Uveoretinitis. Stem Cell Reports 2017; 8:1214-1225.

1. A conditioning protocol for a primary cell culture comprising:
culturing a population of primary cells isolated from a donor tissue or organ in a primary cell culture medium; and
treating the population of primary cells with dim ethyloxalylglycine (DMOG), tumor necrosis factor α (TNF-α), or both DMOG and TNFα by adding the DMOG and/or TNFα to the primary cell culture medium.

2. The conditioning protocol of claim 1, wherein the primary cell culture medium comprises a base cell culture medium, human platelet lysate (hPL), vitamin C, and glutathione.

3. The conditioning protocol of claim 1, wherein the concentration of DMOG added to the primary cell culture is approximately 1 mM.

4. The conditioning protocol of claim 1, wherein the concentration of TNF-α added to the primary cell culture is approximately 10 ng/ml.

5. The conditioning protocol of claim 1, wherein the population of primary cells comprises a population of mesenchymal stem cells (MSC), and wherein the donor tissue or organ is a pancreas, bone marrow, blood, adipose tissue, lung, heart, umbilical cord, or placenta.

6. The conditioning protocol of claim 5, wherein the population of primary cells comprises a population of intra-pancreatic tissue derived (IPTD) MSCs, and wherein the donor tissue or organ is a pancreas.

7. The conditioning protocol of claim 1, wherein the population of primary cells comprises a population of islet cells isolated from a donor pancreas.

8. The conditioning protocol of claim 1, wherein the donor organ or tissue is a cadaver donor organ or tissue, an autologous donor organ or tissue, or an allogenic donor organ or tissue.

9. A method of generating one or more populations of primary cells for therapeutic application, the method comprising:
isolating a first population of primary cells from a donor tissue or organ; and
performing the conditioning protocol of claim 1 on the first population of primary cells.

10. The method of claim 9, further comprising:
isolating a second population of primary cells from a donor tissue or organ; and
performing the conditioning protocol of claim 1 on the second population of primary cells.

11. The method of claim 9, wherein the first population of primary cells comprises a population of mesenchymal stem cells, and wherein the donor tissue or organ is a pancreas, bone marrow, blood, adipose tissue, lung, heart, umbilical cord, or placenta.

12. The method of claim 11, wherein the first population of primary cells comprises a population of IPTD-MSCs, and wherein the donor tissue or organ is a pancreas.

13. The method of claim 10, wherein the second population of primary cells comprises a population of islet cells, and wherein the donor tissue or organ is a pancreas.

14. The method of claim 9, wherein the first population of primary cells and the second population of primary cells are isolated from a single donor tissue or organ.

15. A method of generating a mixture of cells for islet transplantation comprising:
isolating a population IPTD-MSCs and a population of islet cells from a single donor pancreas;
separately performing the conditioning protocol of claim 1 on the population IPTD-MSCs and the population of islet cells;
combining the conditioned IPTD-MSCs and the islet cells to form the mixture of cells for transplanting into a subject.

16. The method of claim 15, wherein the single donor pancreas is a cadaver donor pancreas, an autologous donor pancreas, or an allogenic donor pancreas.

17. A method for treating a disease or condition comprising co-transplanting a therapeutically effective amount of the mixture of cells of claim 15 into a subject.

18. The method of claim 17, wherein the mixture of cells is co-transplanted into the liver of the subject via infusion or injection.

19. The method of claim 17, wherein the disease or condition is type 1 diabetes or pancreatitis.

20. The method of claim 18, wherein the mixture of cells is co-transplanted into the liver of the subject via intravenous administration, catheter administration, or intraperitoneal administration.
